(19) United States
(12) Patent Application Publication
(10) Pub. No.: US 2005/0260137 A1
(43) Pub. Date: Nov. 24, 2005

(54) Contrast agents for magnetic resonance imaging

(75) Inventors: Havva Yagci Acar, Istanbul (TR); Faisal Ahmed Syud, Clifton Park, NY (US); Rachel Nicole Garaas, Clifton Park, NY (US); Peter John Bonitatebus JR., Guilderland, NY (US); Amit Mohan Kulkarni, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/889,618

(22) Filed: Jul. 12, 2004

Related U.S. Application Data
(60) Provisional application No. 60/572,726, filed on May 18, 2004.

(51) Int. Cl. A61K 49/00

(52) U.S. Cl. 424/9.34; 424/9.36

(57) ABSTRACT

A contrast agent for magnetic resonance imaging having a plurality of nanoparticles. Each of the nanoparticles has: a signal generating core having a diameter of up to 10 nm; at least one organic layer of at least one of a polymer, a monomer, and a surfactant; and a water soluble outer shell of at least one of a polymer, a monomer, and a ligand. The organic layer is adsorbed upon and substantially surrounds and stabilizes the signal generating core. The water soluble outer shell solubilizes and provides biocompatibility for each of the nanoparticles. The contrast agents provide enhanced relaxivity, high signal-to-noise ratios, and targeting abilities. In addition, the contrast agents possess resistance to agglomeration, controlled particle size, blood clearance rate, and biodistribution. Methods of making such contrast agents and nanoparticles are also disclosed.

### CROSS-REFERENCE TO RELATED APPLICATION

This application claims the benefit of U.S. Provisional Application No. 60/572,726, filed May 18, 2004.

### BACKGROUND OF THE INVENTION

The present invention relates generally to the field of magnetic resonance imaging (MRI) contrast agents comprising a plurality of magnetic nanoparticles. More particularly, the present invention relates to the design and synthesis of magnetic nanoparticles comprising a monocrystalline superparamagnetic core coated with an organic shell and decorated with targeting moieties.

Diagnostic imaging procedures and contrast agents are used to study organs, tissues, and diseases in a body. MRI is most effective at providing images of tissues and organs that contain water, such as the brain, internal organs, glands, blood vessels, and joints. Magnetic resonance imaging is based on the magnetic properties of atoms. When focused radio wave pulses are broadcast towards aligned hydrogen atoms in a tissue of interest, the hydrogen atoms return a signal as a result of proton relaxation. The subtle differences in the signal from various body tissues enable MRI to differentiate between organs, and potentially contrast benign and malignant tissues. MRI is useful for detecting tumors, bleeding, aneurysms, lesions, blockage, infection, joint injuries, and the like.

In in-vivo diagnostics, MR imaging provides good resolution characteristics, but has poor sensitivity when compared to other imaging techniques. The administration of contrast agents greatly improves imaging sensitivity. Contrast agents function by changing the relaxation time of a tissue that they occupy by enhancing the relaxation time of the water protons in a close range due to time-dependent magnetic dipolar interaction between the magnetic moments of the contrast agent and the water protons.

Contrast agent specificity is another desired property for enhancing contrast at a site of interest and providing functional information through imaging. Natural bio-distribution of contrast agents depends upon the size, charge, surface chemistry, and administration route. Contrast agents may concentrate in either healthy tissue or at lesion sites and increase the contrast between the normal tissue and the lesion. In order to maximize contrast, it is necessary to concentrate as much of the contrast agent at the site of interest as possible.

Contrast agents comprising one or more crystalline superparamagnetic iron oxide nanoparticles embedded in an organic coating are currently known. These nanoparticles generally have sizes in the range of 50-400 nm and have been evaluated for magnetic separation, cell tracking, and imaging. Magnetic nanoparticles in the 20-160 nm size range have been tested for clinical applications, such as MRI contrast agents for liver and spleen imaging, bowel contrast, and MR angiography. Most of these contrast agents are based upon dextran or dextran derivatives as coating materials. Dextran, however, may induce anaphylactic reactions.

Iron oxide nanoparticles are typically synthesized and precipitated in alkaline aqueous solutions, and tend to have a broad size distribution. Extensive manufacturing techniques, including multiple purification and size separation steps, are necessary to obtain the desired sizes and size distributions. The size of the iron oxide nanoparticles directly relates to the superparamagnetism and the relaxivity of the contrast agent. In addition, nanoparticles obtained using current methods also have a low level of crystallinity, which significantly impacts the sensitivity of the contrast agent. Moreover, nanoparticles tend to agglomerate, due to weak and reversible adsorption of the coating material on the magnetic crystal surface and strong interparticle interactions. Aggregation increases the size of the nanoparticle, resulting in rapid blood clearance as well as reducing targeting efficiency.

Contrast agents have inherent problems that limit targeting efficiency, such as large particle sizes, tendency to agglomerate, quick blood clearances, low efficiency of ligand attachment, and the accessibility of ligands to the biomarker targets. Therefore, what is needed is a contrast agent having a particle size that is sufficiently small to avoid rapid clearance from the blood. What is also needed is a contrast agent having a particle size that is capable of imaging organs other than those of the reticuloendothelial system (RES) and to achieve receptor-directed delivery of the contrast agent. What is further needed is a contrast agent that is able to detect the increased presence of chemical biomarkers and provide biochemical information on the early presence of a specific disease state. What is also needed is a contrast agent comprising coated nanoparticles that are resistant to agglomeration. Finally, what is needed is a simple method to provide small iron oxide nanoparticles without excessive size selection steps.

### SUMMARY OF INVENTION

The present invention meets these and other needs by contrast agents based on superparamagnetic iron oxide in a core-shell structure. The contrast agents provide enhanced relaxivity, high signal-to-noise ratios, and targeting abilities. In addition, the contrast agents possess resistance to agglomeration, controlled particle size, blood clearance rate, and biodistribution. A nanoparticle having a signal generating core and a stabilizing coating is also disclosed. Methods of making such contrast agents and nanoparticles are also disclosed.

Accordingly, one aspect of the invention is to provide a contrast agent comprising a plurality of nanoparticles. Each of the plurality of nanoparticles comprises: a signal generating core having a diameter of up to 10 nm; at least one organic layer comprising at least one of a polymer, a monomer, and a surfactant; and a water soluble outer shell comprising at least one of a polymer, a monomer, and a ligand. The at least one organic layer is adsorbed upon and substantially surrounds the signal generating core, and stabilizes the signal generating core. The water soluble outer shell solubilizes each of the plurality of nanoparticles and provides biocompatibility for each of the plurality of nanoparticles.

A second aspect of the invention is to provide a nanoparticle. The nanoparticle comprises: a signal generating core having a diameter of up to 10 nm and a stabilizing coating disposed on and substantially covering the signal generating core. The stabilizing coating comprises: an inner shell comprising at least one of a polymer, a monomer, and a surfactant, wherein the inner shell is adsorbed upon and substantially surrounds the signal generating core and stabilizes the signal generating core; and a water soluble outer shell disposed on an outer surface of the inner shell and substantially surrounding the inner shell. The water soluble outer shell comprises at least one of a second polymer, a second monomer, and a ligand. The water soluble outer shell solubilizes the nanoparticle.

A third aspect of the invention is to provide a contrast agent comprising a plurality of nanoparticles. Each of the plurality of nanoparticles comprises: a signal generating core having a diameter of up to 10 nm, wherein the signal generating core is superparamagnetic; and a stabilizing coating disposed on and substantially covering the signal generating core. The stabilizing coating comprises: an inner shell comprising at least one of a polymer, a monomer, and a surfactant, wherein inner shell is adsorbed upon and substantially surrounds the signal generating core, and wherein inner shell stabilizes the signal generating core; and a water soluble outer shell. The water soluble outer shell is disposed on an outer surface of the inner shell and substantially surrounds the inner shell. The water soluble outer shell comprises at least one of a second polymer, a second monomer, and a second ligand. The water soluble outer shell solubilizes each of the plurality of nanoparticles and provides biocompatibility for each of the plurality of nanoparticles.

A fourth aspect of the invention is to provide a method of making a plurality of monodisperse nanoparticles, wherein each of the plurality of nanoparticles comprises a substantially crystalline signal generating core having a diameter of up to 10 nm and a stabilizing coating disposed on and substantially covering the signal generating core. The stabilizing coating comprises: an inner shell comprising at least one of a polymer, a monomer, and a surfactant, wherein the inner shell is adsorbed upon and substantially surrounds the signal generating core and stabilizes the signal generating core; and a water soluble outer shell disposed on an outer surface of the inner shell and substantially surrounding the inner shell. The method comprises the steps of: providing the signal generating core and the at least one polymerizable layer, wherein the at least one polymerizable layer is adsorbed upon and substantially surrounds the signal generating core, and wherein the at least one polymerizable layer stabilizes the signal generating core; forming the water soluble shell on an outer surface of the at least one polymerizable layer, wherein the water soluble outer shell solubilizes and provides biocompatibility for each of the plurality of nanoparticles; and covalently bonding the at least one polymerizable layer to the water soluble shell.

These and other aspects, advantages, and salient features of the present invention will become apparent from the following detailed description, the accompanying drawings, and the appended claims.

### BRIEF DESCRIPTION OF DRAWINGS

FIG. 1 is a schematic representation of a nanoparticle comprising the contrast agent of the present invention;

FIG. 2 is a schematic representation of a nanoparticle of the present invention;

FIG. 3 is a schematic representation of the linking of the inner and outer layers of the nanoparticle of the present invention;

FIG. 4 is a table summarizing the properties of the contrast agent of the present invention;

FIG. 5a, 5b, 5c, and 5d are magnetic resonance images of a mouse before injection and a specified time after injection of contrast agents of the present invention;

FIG. 6 is a schematic representation of the organic layer of the present invention comprising a monomer having a surface binding head group and at least one polymerizable functionality; and

FIG. 7 is a schematic representation of the water soluble outer shell of the present invention comprising a ligand comprising a water soluble polymer attached to a polymerizable hydrocarbon moiety.

### DETAILED DESCRIPTION

In the following description, like reference characters designate like or corresponding parts throughout the several views shown in the figures. It is also understood that terms such as “top,” “bottom,” “outward,” “inward,” and the like are words of convenience and are not to be construed as limiting terms.

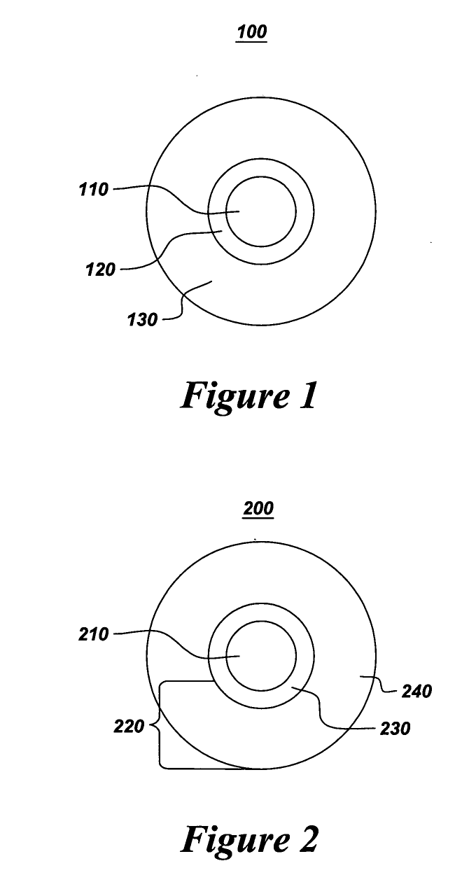

Referring to the drawings in general and to FIG. 1 in particular, it will be understood that the illustrations are for the purpose of describing a preferred embodiment of the invention and are not intended to limit the invention thereto. The present invention provides a contrast agent for the imaging of biological tissues. The contrast agent comprises a plurality of nanoparticles. Each of the plurality of nanoparticles is designed to enhance signal and contrast, and to provide prolonged blood circulation time and targeted delivery of the contrast to specific organs, tissues, disease states, and the like. Further, the contrast agents may be designed to maximize contrast with respect to physiological parameters of interest, such as pH and temperature, which are important indicators of abnormality and disease.

A schematic cross-sectional view of a single such nanoparticle 100 is shown in FIG. 1. Nanoparticle 100 comprises a signal generating core 110, at least one organic layer 120 that is adsorbed upon and substantially surrounds signal generating core 110, and a water soluble outer shell 130, that solubilizes and provides biocompatibility for nanoparticle 100.

Contrast agents function by changing the relaxation time of a tissue that they occupy. Contrast agents for MR are typically magnetic materials that enhance the relaxation time of water protons at close range due to time-dependent magnetic dipolar interaction between the magnetic moments of the contrast agent and the water protons. The efficiency to shorten relaxation times of protons is defined as relaxivity R, which is inversely proportional to the relaxation time T. MR contrast agents may be either positive agents (also referred to hereinafter as “T1 agents”) that illuminate or “light up” the tissue that they occupy, or negative agents (also referred to hereinafter as “T2 agents”) that make a tissue appear darker. Positive T1 agents have a relaxivity R1, where R1=1/T1, whereas negative T2 agents have a relaxivity R2, where R2=1/T2. Examples of T1 agents include, but are not limited to, paramagnetic gadolinium species, such as Gd-DTPA and the like. Non-limiting examples of T2 agents include superparamagnetic iron oxide nanoparticles. Superparamagnetic agents provide higher relaxivities than paramagnetic agents, as they generally have magnetic moments that are about 100 times greater than those of paramagnetic agents.

Particulate iron oxide-based contrast agents that are larger than 50 nm are cleared from the blood rapidly by the uptake of the macrophages of the reticuloendothelial system (RES), and are thus used as contrast agents for organs that comprise the RES system, such as the like liver, spleen, and bone marrow. Iron oxide-based contrast agents that are smaller than 50 nm are used to image other organs or to achieve receptor-directed delivery of the contrast agent.

Nanoparticle 100 has a diameter of up to about 100 nm. In one embodiment, nanoparticle 100 has a diameter of up to about 50 nm. In a preferred embodiment, nanoparticle 100 has a diameter in a range from about 10 nm to about 30 nm.

Signal generating core 110 enhances the relaxation time of the water protons in a close range due to time-dependent magnetic dipolar interaction between the magnetic moments of the contrast agent and the water protons. Signal generating core 110 has a diameter of up to about 30 nm. In one embodiment, signal generating core 110 has a diameter of up to 10 nm. In one particular embodiment, signal generating core 110 has a diameter in a range from about 4 nm to about 10 nm. In one embodiment, signal generating core 110 is a monodisperse superparamagnetic nanoparticle and comprises at least one of: an iron oxide, such as but not limited to, hematite (Fe₂O₃), ferrite (Fe₃O₄), or magnetite; a mixed spinel ferrite having the general formula MFe₂O₄, where M is a metal such as, but not limited to, manganese, cobalt, copper, nickel, gadolinium, zinc, and vanadium; and combinations thereof. One non-limiting example of such a synthetic route is disclosed in U.S. patent application Ser. No. 10/208,946, filed on Jul. 31, 2002, by Peter John Bonitatebus and Havva Acar, entitled “Nanoparticle having an Inorganic Core,” the contents of which are incorporated herein by reference in their entirety. In one embodiment, signal generating core 110 is substantially crystalline. In this context, “substantially crystalline” is understood to mean that signal generating core 110 comprises at least 50 volume percent and, preferably, at least 75 volume percent crystalline material. Most preferably, signal generating core 110 is a single crystal.

Superparamagnetic behavior is a size-dependent phenomenon, and the efficiency of the contrast agent depends on the size and the size distribution of the signal generating core 110. Thus, the small size and narrow size distribution of signal generating core 110 enhances the magnetic signal and its homogeneity.

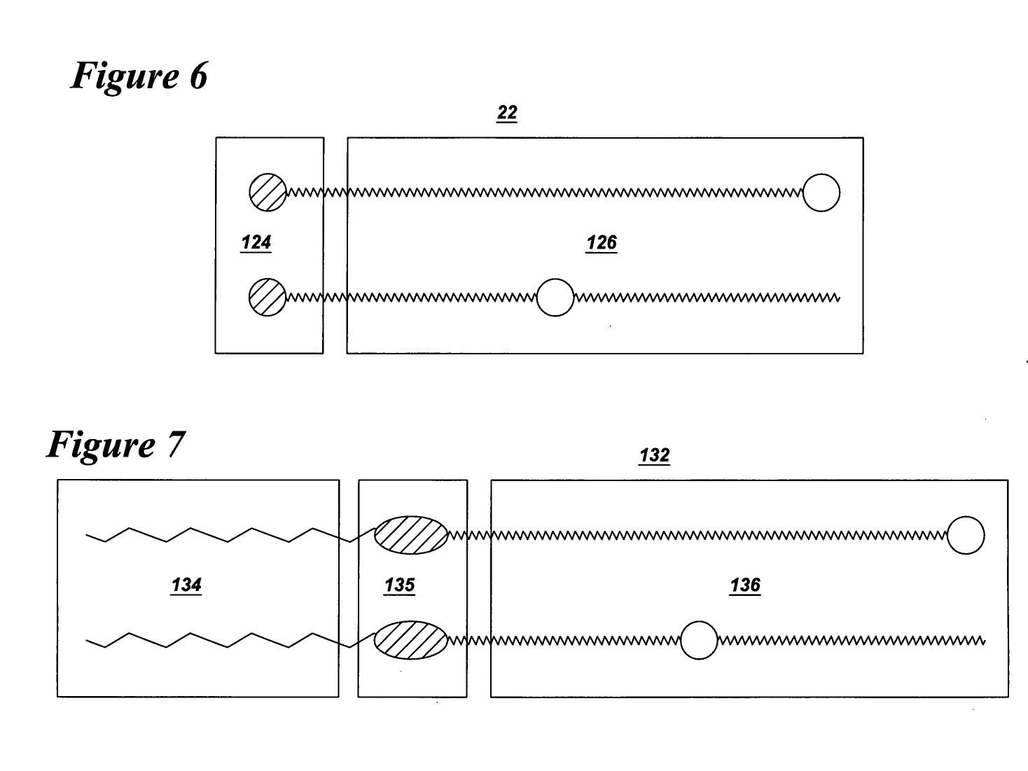

The at least one organic layer 120 surrounds and entraps signal generating core 110, prevents the aggregation of a plurality of signal generating cores 110, and enhances the stability of each signal generating core 110. Aggregation of the plurality of signal generating cores 110 adversely affects the relaxivity. The at least one organic layer 120 comprises at least one of a polymer, a monomer, a ligand, and a surfactant, and has a thickness in a range from about 0.1 nm to about 100 nm. The at least one organic layer 120 may be chemisorbed onto an outer surface of signal generating core 110 during the synthesis of signal generating core 110. In one embodiment, the at least one organic layer 120 comprises a water soluble surface binding polymer and a hydrophobic polymer. In another embodiment, the at least one organic layer 120 comprises a copolymer of a monomer having a pendant group that has an affinity for, and adsorbs onto, the outer surface of signal generating core 110 and a hydrocarbon group, wherein the hydrocarbon group includes a carbon chain of at least three carbon atoms. Non-limiting examples of such copolymers include copolymers of acrylic acid, undecenoic acid, lauryl acrylate, combinations thereof, and the like. Specific examples of such copolymers include polyacrylic acid, poly(undecenoic acid), and poly(lauryl acrylate). In yet another embodiment, shown in FIG. 6, the at least one organic layer 120 comprises a monomer 122 having a surface binding head group 124 and at least one polymerizable functionality 126, such as, but not limited to, undecenoic acid. In one non-limiting example, the surface binding head group is an ionic head group. In one non-limiting example, the surface binding head group is a trialkoxy silane.

In one embodiment, the at least one organic layer 120 comprises a surfactant having a surface binding head group and at least one hydrocarbon tail. The surfactant may be one of lauric acid and sodium dodecyl sulfate.

Water soluble outer shell 130 comprises at least one of a polymer, a monomer, and a ligand, and has a thickness in a range from about 0.1 nm to about 100 nm. Contrast agent 100 may be directed towards specific organs or sites by tailoring the size, polarity and charge of the water soluble outer shell 130. Water soluble outer shell 130 solubilizes nanoparticle 100 in an aqueous medium, provides biocompatibility for nanoparticle 100, and may, in some instances, affect the pharmokinetics. Water soluble outer shell 130 enables the contrast agent to bind to a specific site through molecular recognition of a portion of water soluble outer shell 130 by a specific biomarker, also known as a “receptor.” In one embodiment, water soluble shell 130 further includes at least one targeting moiety such as, but not limited to, at least one of a peptide comprising, for, example LSIPKKA, an antibody, at least one sugar, at least one organic molecule such as folic acid, estradiol, combinations thereof, and the like.

In another embodiment, water soluble outer shell 130 comprises a copolymer of a carboxylic acid and a hydrocarbon, wherein the hydrocarbon includes a carbon chain of at least three carbon atoms. Non-limiting examples of such copolymers include copolymers of acrylic acid, undecenoic acid, lauryl acrylate, combinations thereof, and the like. Specific examples of such copolymers include polyacrylic acid, poly(undecenoic acid), and poly(lauryl acrylate).

In yet another embodiment, water soluble outer shell 130 comprises a monomer having a surface binding head group and at least one polymerizable functionality. One non-limiting example of such a monomer is undecenoic acid. In a third embodiment, water soluble outer shell 130 comprises at least one ligand. Alternatively, water soluble outer shell 130 includes a ligand 132 comprising a water soluble polymer 134, such as polyethylene glycol (PEG), attached to a polymerizable hydrocarbon moiety 136 such as, for example, undecylene, via linkage 135 wherein the hydrocarbon moiety comprises a chain of at least three carbon atoms, as shown in FIG. 7.

The invention also provides a nanoparticle that may be used in imaging applications in addition to MRI contrast agents. The nanoparticle 200, which is shown in FIG. 2, comprises: a signal generating core 210 having a diameter of up to about 10 nm; a stabilizing coating 220 disposed on and substantially covering the signal generating core 210. The stabilizing coating comprises an inner shell 230 and a water soluble outer shell 240. The inner shell 230 is adsorbed upon and substantially surrounds and stabilizes the signal generating core 210. The inner shell 230 comprises at least one of a polymer, a monomer, and a surfactant. The water soluble outer shell 240 is disposed on an outer surface of the inner shell 230 and substantially surrounds the inner shell 230. The water soluble outer shell 240 solubilizes the nanoparticle in an aqueous medium. In one embodiment, water soluble outer shell 240 provides biocompatibility for the nanoparticle 220. Water soluble outer shell 240 comprises at least one of a second monomer and a ligand.

Signal generating core 210 has a diameter of up to 30 nm. In one embodiment, signal generating core 210 has a diameter of up to 10 nm. In one particular embodiment, signal generating core 210 has a diameter in a range from about 4 nm to about 10 nm. In one embodiment, signal generating core 210 is superparamagnetic and comprises an iron oxide, as previously described hereinabove. Signal generating core 210 may further comprise at least one of gadolinium, manganese, copper, nickel, cobalt, zinc, germanium, gold, silver, compounds comprising group II (A or B) and group VI elements (such compounds are also referred to hereinafter as “II-VI compounds”), compounds comprising group IV and group VI elements (also referred to hereinafter as “IV-VI compounds”), combinations thereof, and the like. Alternatively, signal generating core 210 is responsive to laser radiation and comprises at least one of gold, silver, combinations thereof, and the like. In yet another embodiment, signal generating core 210 is radio-opaque, and comprises at least one of gadolinium, barium, combinations thereof, and the like.

Inner shell 230 comprises at least one of a polymer, a monomer, and a surfactant and has a thickness in a range from about 0.1 nm to about 100 nm. In one embodiment, inner shell 230 comprises a water soluble surface binding polymer and a hydrophobic polymer. In another embodiment, inner shell 230 comprises a copolymer of a monomer, the monomer having a pendant group that has an affinity for, and adsorbs onto, the outer surface of signal generating core 210 and a hydrocarbon group, wherein the hydrocarbon group includes a carbon chain of at least three carbon atoms. Non-limiting examples of such copolymers include copolymers of acrylic acid, undecenoic acid, lauryl acrylate, combinations thereof, and the like. Inner shell 230 may comprise polyacrylic acid, poly(undecenoic acid), lauryl acrylate, and combinations thereof. In yet another embodiment, inner shell 230 comprises a monomer having a surface binding head group and at least one polymerizable functionality, such as, but not limited to, undecenoic acid. Alternatively, inner shell 230 comprises a monomer having a surface binding functionality and at least one polymerizable functionality. In one non-limiting example, the surface binding head group is a trialkoxy silane.

In one embodiment, inner shell 230 comprises a surfactant having a surface binding head group and at least one hydrocarbon tail. The surfactant may be one of lauric acid and sodium dodecyl sulfate. In another embodiment, the at least one organic layer includes a ligand comprising a water soluble polymer attached to a polymerizable hydrocarbon moiety, wherein the hydrocarbon moiety comprises a chain of at least three carbon atoms.

Water soluble outer shell 240 comprises at least one of a polymer, a monomer, and a ligand has a thickness in a range from about 0.1 nm to about 100 nm. Water soluble outer shell 240 solubilizes nanoparticle 200 and, in one embodiment, provides biocompatibility for nanoparticle 200. In one embodiment, water soluble outer shell 240 further includes at least one targeting moiety such as, but not limited to, at least one of a peptide, a protein, an antibody, a sugar, at least one molecule of biological significance such as, folic acid, estradiol, combinations thereof, and the like.

In one embodiment, water soluble outer shell 240 comprises a copolymer of a carboxylic acid and a hydrocarbon, the hydrocarbon having a carbon chain of at least three carbon atoms. Non-limiting examples of such copolymers include copolymers of acrylic acid, undecenoic acid, lauryl acrylate, combinations thereof, and the like. Specific examples of such copolymers include polyacrylic acid, poly(undecenoic acid), and lauryl acrylate. In yet another embodiment, water soluble outer shell 240 comprises a monomer, the monomer having a surface binding head group and at least one polymerizable functionality. One non-limiting example of such a monomer is undecenoic acid. In a third embodiment, water soluble outer shell 240 comprises a ligand comprising a water soluble polymer, such as PEG, attached to a polymerizable hydrocarbon moiety such as, for example, undecylene, wherein the hydrocarbon moiety comprises a chain of at least three carbon atoms. In one particular embodiment, inner shell comprises a surface binding head group and at least one polymerizable functionality, such as undecenoic acid, and water soluble outer shell 240 includes a ligand comprising a water soluble polymer, such as PEG, attached to a polymerizable hydrocarbon moiety, such as undecylene.

Particle 200 has a diameter of up to about 100 nm. In one embodiment, nanoparticle 200 has a diameter of up to about 50 nm and, in a preferred embodiment, has a diameter in a range from about 10 nm to about 30 nm.

Another aspect of the invention is to provide a method of making both the contrast agent and the monodisperse nanoparticle described hereinabove. The method comprises the steps of: providing the signal generating core having at least one polymerizable layer disposed on its outer surface; forming a water soluble shell on an outer surface of the at least one polymerizable layer; and covalently bonding the at least one polymerizable layer to the water soluble shell.

In one embodiment, the monodisperse signal generating core is formed by a non-aqueous synthetic route in which at least one organometallic compound is thermally decomposed at high temperatures in a solvent in the presence of a surfactant and an oxidant. Such methods produce superparamagnetic nanoparticles coated with a surfactant monolayer. A non-aqueous synthetic approach tends to produce more spherical nanoparticles. One non-limiting example of such a synthetic route is disclosed in U.S. patent application Ser. No. 10/208,945, filed on Jul. 31, 2002, by Peter John Bonitatebus and Havva Acar, entitled “Method of Making Crystalline Nanoparticles,” the contents of which are incorporated herein by reference in their entirety. The method comprises first combining a nonpolar aprotic organic solvent, an oxidant, and a first polymerizable surfactant having a surface binding head group. At least one organometallic compound comprising a metal and at least one ligand is then added to the combined nonpolar aprotic organic solvent, oxidant, and first polymerizable surfactant. The combined nonpolar aprotic organic solvent, oxidant, first polymerizable surfactant, and the at least one organometallic compound are then heated under an inert gas atmosphere to a first temperature in a range from about 30° C. to about 400° C. for a first time interval to form the signal generating core surrounded by the first polymerizable surfactant. The signal generating core surrounded by the first surfactant is then precipitated out of the nonpolar solvent. The nanoparticles generated by the non-aqueous synthetic method described above have signal generating core sizes in a range from about 5 nm to about 10 nm. The nanoparticles so formed have a size distribution of less than about 15 percent.

The plurality of signal generating cores coated with the first surfactant is precipitated out of solution by adding at least one of an alcohol or a ketone to the nonpolar aprotic solvent. Alcohols such as, but not limited to, methanol and ethanol may be used. Alcohols having at least three carbon atoms, such as isopropanol, are preferred, as precipitation by the addition of such alcohols tends to produce the least degree of agglomeration of the plurality of nanoparticles. Ketones such as, but not limited to, acetone may be used in conjunction with, or separate from, an alcohol in the precipitation step. Once precipitated, the plurality of signal generating cores coated with the first surfactant can be suspended in water by sonication. The addition of an aqueous solution of similar or different type in a dropwise fashion with continuous sonication at temperatures between about 40° C. to about 60° C. allows the hydrocarbon tails to interact with each other and form a micellar bilayer coating around each of the magnetic signal generating cores. In one embodiment, the first surfactant is undecenoic acid (UD), the second surfactant is undecenoic polyethylene glycol (UDPEG), and the resulting bilayer is UD/UDPEG.

The present invention also provides an aqueous synthetic route for the synthesis of substantially monodisperse crystalline superparamagnetic iron oxide nanoparticles. The aqueous synthesis of iron oxide in the presence of micelle forming, surface binding molecules provides improved control over the size distribution of iron oxide nanoparticles relative to synthetic methods that do not use surface binding surfactants.

The method comprises forming a core-shell structure in which the shell comprises a bilayer of at least one surface binding surfactant. The surfactants have a high affinity for the oxide surface of the iron oxide core. As the iron oxide crystal starts to form, the surfactants adsorb on the crystal surface and thus prevent further crystal growth at the surface. The presence of the coating during crystal growth controls the size distribution of the crystals limits agglomeration and prevents formation of a thick non-magnetic oxidation layer on the crystal surface. Consequently, the nanoparticles formed by this aqueous route have a much higher magnetization than currently available superparamagnetic iron oxide contrast agents. For example, iron oxide nanoparticles coated with an undecenoic acid bilayer and having a diameter of about 8.4 nm have a saturation magnetization of about 95.4 emu/g. The nanoparticles generated by this method have superparamagnetic cores with sizes in a range from about 5 nm to about 10 nm. The nanoparticles have a size distribution of less than about 20 percent and, in one embodiment, the size distribution is less than about 15 percent.

In a typical aqueous-based preparation of a plurality of signal generating cores, NaNO₃, FeCl₂, and FeCl₃.6H₂O are dissolved under nitrogen in deoxygenated Milli-Q water with vigorous stirring. The Fe²⁺/Fe³⁺ molar ratio is about 0.5. The solution is then heated to 80° C., and then charged with NH₄OH solution and a surfactant or monomer, such as undecenoic acid. Crystal growth is allowed to proceed at temperature with constant vigorous stirring to produce a stable colloidal suspension of nanoparticles. The colloidal suspension is then cooled slowly to room temperature with stirring to yield substantially monodisperse spinel-structured mixed iron oxide (γ-Fe₂O₃)_{1-y}(Fe₃O₄)_{y} nanocrystals that are coated with an undecenoic acid bilayer.

The synthesis of the signal generating cores in the presence of surface adsorbing surfactants controls the size and size distribution of the signal generating cores and separates the signal generating cores from each other, allowing each signal generating core to affect a larger volume of water molecules in their immediate vicinity.

The plurality of signal generating cores coated with the first surfactant is isolated by precipitation. Precipitation of the coated signal generating cores is achieved by adding at least one of an alcohol or a ketone to the nonpolar aprotic solvent. Alcohols such as, but not limited to, methanol and ethanol may be used. Alcohols having at least three carbon atoms, such as isopropanol, are preferred, as precipitation by the addition of such alcohols tends to produce lower degrees of agglomeration of the nanoparticles. Ketones such as, but not limited to, acetone may be used in conjunction with, or separate from, an alcohol in the precipitation step. Alternatively, the plurality of signal generating cores coated with the first surfactant are extracted into an aprotic solvent, such as, but not limited to, toluene and chloroform.

Once the signal generating core 110 has been coated with at least one organic layer 120, a water soluble outer shell 130 is then deposited on the stabilized nanoparticles by first mixing the material forming the water soluble outer shell 130 with the isolated nanoparticles of signal generating cores 110 coated with the inner layer 120. The signal generating cores 110 coated with the at least one organic layer 120 are then transferred into an aqueous solution containing the material that forms the water soluble outer shell 130. Alternatively, the signal generating cores coated with the at least one organic layer 120 in organic solvents such as, but not limited to, toluene, are transferred into an aqueous solution containing the material that forms the water soluble outer shell 130. The combined liquids are then centrifuged to transfer the coated cores into the aqueous phase, and the material adsorbs onto the coated core to form the water soluble outer shell 130.

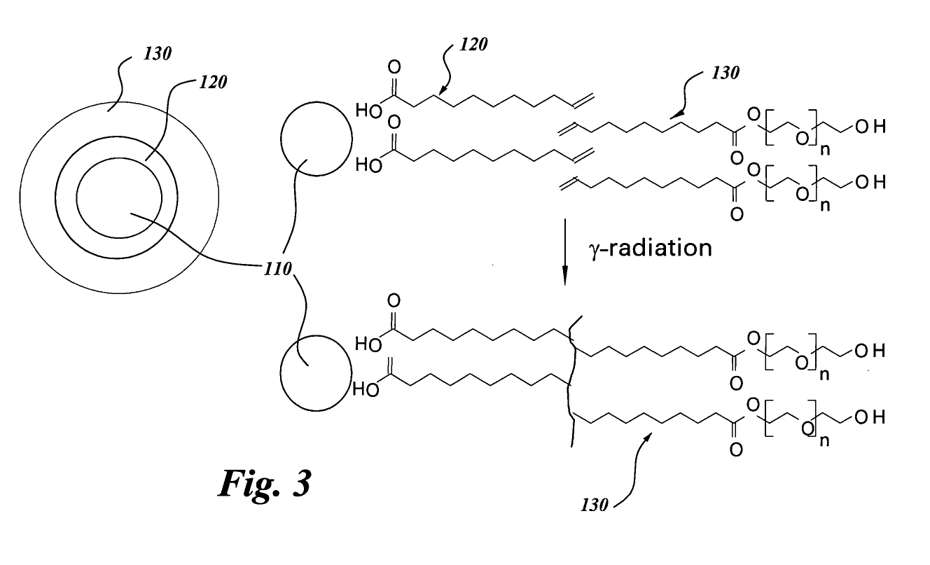

The water soluble outer shell 130 is then covalently bonded—or linked—to the at least one polymerizable layer 120 by at least one of cross-polymerization by gamma irradiation, and heating, as schematically shown in FIG. 3. Covalent bonding fixes the stabilizing inner layer and water soluble outer shell around the core and provides stability for the nanoparticles, making them resistant to agglomeration.

Ligands may possess physiologically responsive entities, such as, but not limited to, polymers that are sensitive to pH or temperature. Non-limiting examples of such polymers include poly(N-isopropyl acrylamide), pluronics, poly(hydroxyethyl methacrylate), and polyacrylic acid. The physiological MRI contrast agents of the present invention may be applied to cells or be administered to a body intravenously and allowed to circulate in the bloodstream. The contrast agents may be used in the form of a suspension in a solvent, such as physiological saline, sugar solution, and the like. In specific applications, at least one pharmacologically acceptable additive, such as a carrier or expedient, may be used. Preferably, the contrast agent is administered in the form of a stable aqueous solution. Additives used vary depending on factors such as the mode of administration, administration route, and the like. Examples of additives for intravenous injections include buffers, antibacterial agents, stabilizers, solubilizers, and excipient that are either used alone or in combination with each other.

The following examples illustrate the various features and advantages offered by the present invention, and in no way are intended to limit the invention thereto.

### EXAMPLE 1

Iron oxide nanocrystals coated with surfactant and monomer, as described hereinabove, were synthesized according to the non-aqueous synthesis route referred to hereinabove. A mixture of trimethylamine-N-oxide, 10-undecenoic acid (or, alternatively, lauric acid or oleoic acid), and deoxygenated dioctyl ether, each individually dehydrated and deoxygenated, was added under an inert atmosphere to a 50 ml 2-neck Schlenk flask. The mixture was homogenized with vigorous stirring and heating to about 100° C. Iron carbonyl (Fe(CO)₅) was then added to the reaction solution, which was at a temperature in a range from about 100° C. to about 105° C., resulting in instantaneous and aggressive reaction. The reaction mixture was then heated to a temperature in a range from about 120° C. to about 130° C. under nitrogen and maintained at temperature for about 1 hour while being vigorously stirred. Additional iron carbonyl (Fe(CO)₅) was then added to the reaction mixture, and the temperature was rapidly increased to about 280° C. to allow the reaction mixture to reflux. After 1 hour of refluxing and stirring at about 280° C., the color of the reaction mixture turned black. The reaction mixture was then cooled to room temperature, and an equal volume amount of isopropyl alcohol was added to the reaction mixture, yielding a black precipitate, which was then separated out by centrifuging and collected by magnetic decantation. Particles were then readily dispersed in toluene and octane to form homogeneous solutions. Crystal structure, composition, and particle size analysis of the powder was obtained by transmission electron microscopy (TEM) imaging, energy dispersive x-ray (EDX) elemental analysis, x-ray absorption spectroscopy (XAS), and selected area electron diffraction/x-ray diffraction (SAED-XRD) crystal symmetry pattern indexing. The powder obtained was found to comprise monodisperse spinel-structured mixed iron oxide (γ-Fe₂O₃)_{1-y}(Fe₃O₄)_{y} nanocrystals, each having a particle size of about 10 nm±1 nm.

### EXAMPLE 2

Monodisperse, bilayer surfactant- or monomer-coated magnetic nanoparticles were synthesized according to the aqueous route described hereinabove. In a typical preparation, NaNO₃, FeCl₂ (anhydrous) and FeCI₂.6H₂O were dissolved in deoxygenated Milli-Q water with vigorous stirring under nitrogen. The Fe²⁺/Fe³⁺ molar ratio in the solution was 0.5. The solution was heated to 80° C. and then charged with rapid sequential injections of NH₄OH solution and 10-undecenoic acid. Crystal growth proceeded for about 45 minutes at 80° C. with constant vigorous stirring, producing a stable colloidal suspension of nanoparticles, which was then cooled slowly to room temperature with stirring. The suspension was placed on a magnet for at least 1 hour, and then filtered to remove any insoluble material. The material obtained was found to comprise monodisperse spinel-structured mixed iron oxide (γ-Fe₂O₃)_{1-y}(Fe₃O₄)_{y} nanocrystals. The average particle size of the nanocrystals an about 8.5±1.2 nm, as determined TEM.

### EXAMPLE 3

In this example, the preparation of nanoparticles having an iron oxide core, an inner layer comprising a monomer, and a water soluble outer shell that includes at least one ligand comprising PEG and undecenoic acid (PEGylated ligands), all of which are disclosed hereinabove, is described. The PEGYlated ligands were first prepared using either PEGs, or alternatively, PEG monomethyl ethers with molecular weights between 300-5,000 g/mol as starting materials. In one instance, PEG (2,000 Da) was dissolved in dry methylene chloride. Trimethyl amine, and dimethylamino pyridine (DMAP) were added to the solution and stirred under nitrogen in an ice bath. 10-undecenoyl chloride diluted with dry methylene chloride was added dropwise to the chilled solution, and the reaction mixture was stirred for about two hours, first in an ice bath and then at room temperature. The reaction mixture was then filtered, diluted with methylene chloride, and washed three times with 0.1N HCl, 0.1N NaOH and brine solution. After drying with anhydrous MgSO₄, the solvent was removed in vacuo, leaving behind an almost colorless liquid product comprising the PEGylated ligand having the formula HO(CH₂CH₂O)ₙCH₂CH₂OC(═O)(CH₂)₈CH=CH₂.

Iron oxide nanocrystalline cores obtained by either the non-aqueous synthesis route (Example 1) or the aqueous synthetic route (Example 2) described hereinabove were precipitated in isopropanol and then suspended in Milli-Q water by sonication at 60° C. A solution of PEGylated ligand in Milli-Q water, comprising about 5 percent of the PEGylated ligand, was then added dropwise to the suspension during continuous sonication at 60° C. until a stable solution was obtained. The suspension was placed on a magnet for about 1 hour. A precipitate, comprising particles that were not coated with the PEGylated ligand, was separated from the stable suspension by magnetic decantation.

### EXAMPLE 4

In this example, a method of isolating the signal generating core and depositing an outer layer comprising a PEGylated ligand is described. A solution obtained from Example 2 was shaken with chloroform. The organic phase was separated and the chloroform was evaporated under reduced pressure, leaving a residue. The residue was dried further under vacuum at 60° C. for 2 h. The residue was then suspended in water by sonication at 60° C. and 4 mL of 0.4M UDPEG750 solution in milli-Q water was added dropwise to the suspension during continuous sonication until a stable solution was obtained. The final suspension was placed on a magnet for 1 h to remove any precipitate from the stable suspension. The particle size of the coated nanoparticles, as determined by dynamic light scattering (DLS), was 35 nm.

### EXAMPLE 5

In this example, a method isolating the signal generating core and depositing an outer layer comprising a PEGylated ligand is described. A solution obtained from Example 2 was sonicated at 60° C. with toluene for 30 min. The organic phase was separated and added to an aqueous solution of UDPEG750 in a centrifuge tube and centrifuged at 15,000 rpm for 1.5 h. The organic layer was removed and the aqueous layer was sonicated for about 20 min. The stable suspension was then filtered from 20 nm filter and the particle size was 21 nm, as measured by DLS (0.27 mM Fe).

### EXAMPLE 6

The polymerization of the at least one organic layer and the water soluble outer shell, both of which are described hereinabove, is described. Stable nanoparticles, each comprising a signal generating core, at least one organic layer, and a water soluble outer shell, were freeze-dried and then irradiated by gamma radiation to form a polymerized PEGylated coating from the at least one organic layer and outer shell. The total dose of radiation was in a range from 3 Mrad to 5 Mrad. The degree of polymerization due to irradiation was determined by Gel-Permeation Chromatography (GPC). Results indicated the presence of polymers with 4 to 72 repeat units of undecenoic acid (PEG degrades during GPC sample preparation). Hydrodynamic size of the nanoparticles was determined by DLS. For the nanoparticles having polymerized PEGylated coatings, a hydrodynamic diameter (Dh) in a range from about 20 nm to about 80 nm was measured.

### EXAMPLE 7

The removal of excess ligand from the nanoparticle is described. Excess ligand was removed from the nanoparticles after polymerization by ultracentrifugation using ultracentrifuge tubes with 100,000 Da cut off. Alternatively, excess ligand was removed from the nanoparticles after polymerization by centrifugation at 50,000 rpm for 3 h, followed by magnetic decantation. Particle sizes before and after the removal of excess ligand remained the same, indicating stability and resistance to aggregation.

### EXAMPLE 8

In this example, the attachment of targeting moieties to the nanoparticles, as disclosed hereinabove, is described. Targeting moieties may be attached to PEGylated ligands through, for example, free —OH or —NH₂ chain ends, if either hydroxy- or amine-terminated PEG is used in either the at least one organic layer or the water soluble outer shell. Alternatively, a free —OH chain end is converted to an activated carboxylic acid in order to achieve facile conjugation of the targeting moieties to the PEGylated surfactants. For example, 2.25 g of HO(CH₂CH₂O)ₙC(O)(CH₂)₈CH═CH₂, wherein PEG is 2,000 Da, was dissolved in 25 ml of dry dioxane. 1.05 g of N,N′-disuccinimidyl carbonate in 10 ml dry acetone and 0.76 g DMAP in 10 ml dry acetone were then added to the solution. The reaction mixture was stirred at room temperature under nitrogen for 2 days. A white product comprising SucC(O)O(CH₂CH₂O)ₙC(O)(CH₂)₈CH═CH₂ was obtained after precipitation into diethyl ether from acetone.

### EXAMPLE 9

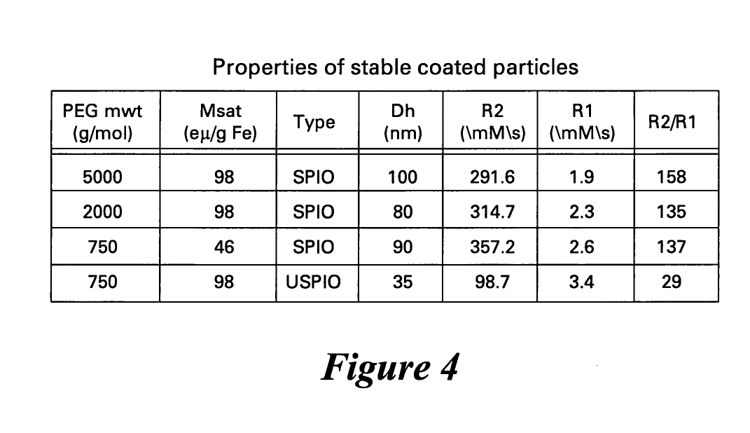

The magnetic properties and imaging capabilities of the contrast agents disclosed hereinabove will now be described. The nanoparticles comprising the contrast agent of the present invention have a high magnetic moment in the presence of a magnetic field and a negligible magnetic moment in the absence of a magnetic field. Magnetization of the nanoparticles was measured using a vibrating sample magnetometer with fields up to 2,500 Gauss at 25° C. The nanoparticles have a saturation magnetization in the range of about 40 emu/g to about 105 emu/g of metal. The saturation magnetization values of nanoparticles having different hydrodynamic diameters (Dh) are listed in FIG. 4.

Magnetic resonance (MR) contrast agents work by shortening the proton relaxation times and hence increasing the contrast and overall image quality. The nanoparticles were found to affect both the longitudinal relaxation (T1) and transverse relaxation times (T2). The relaxation times were measured by imaging nanoparticle suspensions at different concentrations in a 1.5 Tesla scanner at 25° C. The nanoparticles exhibited a longitudinal relaxation rate (R1) in a range from about 1 mM/s to about 10 mM/s, and a transverse relaxation rate (R2) in a range from about 90 mM/s to about 400 mM/s. Values of R1 and R2 that were obtained for nanoparticles are listed in FIG. 4. Nanoparticles having sizes in the range from 50 nm to 100 nm had R2 relaxivity values between 290 mM⁻¹s⁻¹ and 360 mM⁻¹s⁻¹, which is three times greater than values exhibited by currently available contrast agents. The R2/R1 ratios for the nanoparticles were between 135 mM⁻¹s⁻¹ and 158 mM⁻¹s⁻¹, which is about 10 times greater than values exhibited by currently available contrast agents. This indicates a the contrast agent of the present invention acts as a strong T2 contrast agent, providing greater signal intensity and contrast with significantly lower doses than currently available contrast agents. Nanoparticles of the present invention having sizes of less than 50 nm, and, in one embodiment, sizes of less than 35 nm, exhibit R2 relaxivities that are comparable to those of existing contrast agent systems having much greater particle sizes. This result indicates that the smaller nanoparticles of the present invention possess improved magnetics and coatings.

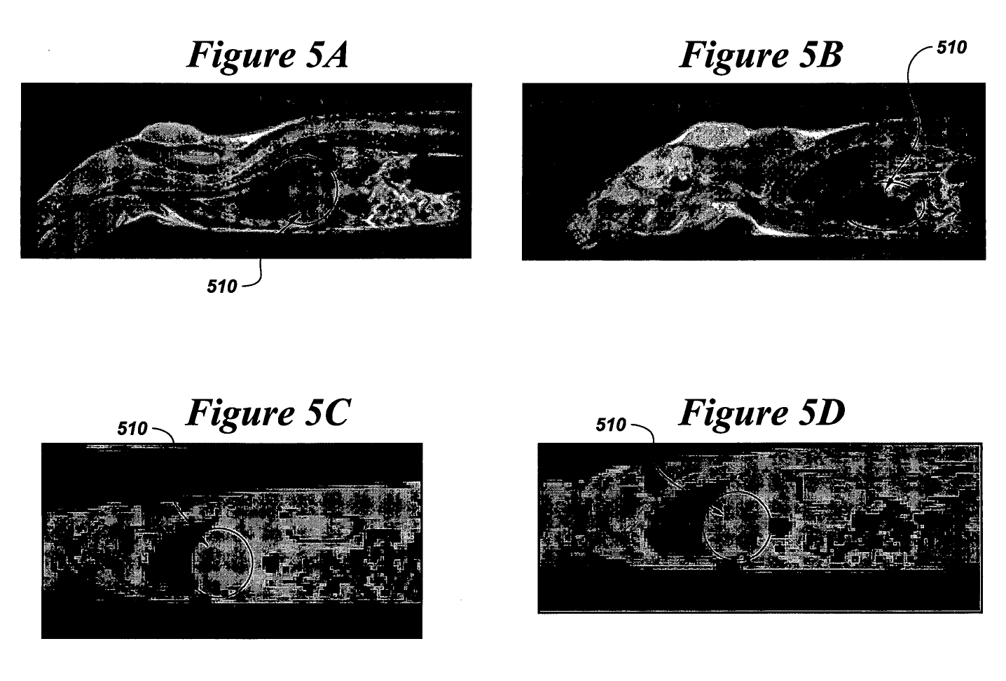

The application of the contrast agents of the present invention in MR applications was evaluated by performing in vivo studies on mice and rats. Solutions of nanoparticles described hereinabove were prepared in 0.9M NaCl at 0.2-5 mg/ml. The animals were injected with known quantities (20-40 micromol Fe/kg body weight) of the nanoparticles and imaged using a 1.5T scanner. In addition, the blood half-life of contrast agents of the present invention was measured by injecting known amounts of nanoparticles into rats and removing blood samples at known time intervals. The blood samples were analyzed using MR imaging to determine the metal concentration at different times after injection. MR images before and after injection were compared to determine the effect of nanoparticles on specific tissues or organs. FIGS. 5a, 5b, 5c, and 5d are T2 weighted MR images of a mouse before (FIGS. 5a and 5c) and a specified time after injection (FIGS. 5b and 5d) for contrast agents having hydrodynamic diameters of 70 nm (UD inner and PEG oleate outer layer) and 35 nm (UD inner and UDPEG outer layer), respectively. After 10 minutes, liver 510 had taken up a sufficient portion of the contrast agent having a hydrodynamic diameter of 75 nm, causing the MR image of liver 510 to noticeably darken (FIG. 5b). The blood half-life of the contrast agent having a hydrodynamic diameter of 70 nm was about 16 minutes. The uptake of the contrast agent having a hydrodynamic diameter of 35 nm by the liver 510 was significantly slower, as evidenced by the absence of darkening of liver 510 after 10 minutes (FIG. 5d), indicating that the decrease in contrast agent particle size had caused the blood half-life to significantly increase. Contrast agents having sizes in the range from about 10 nm to about S0 nm may be used as T1 blood pool agents, and may be decorated for receptor-mediated delivery to target tissues. Entities such as peptides, antibodies, folic acid, estradiol can be attached to the ligand for this purpose.

While typical embodiments have been set forth for the purpose of illustration, the foregoing description should not be deemed to be a limitation on the scope of the invention. Accordingly, various modifications, adaptations, and alternatives may occur to one skilled in the art without departing from the spirit and scope of the present invention.

1. A contrast agent, said contrast agent comprising a plurality of nanoparticles, wherein each of said plurality of nanoparticles comprises:
a) a signal generating core, said signal generating core having a diameter of up to 10 nm;
b) at least one organic layer, said at least one organic layer comprising at least one of a polymer, a monomer, and a surfactant, wherein said at least one organic layer is adsorbed upon and substantially surrounds said signal generating core, and wherein said at least one organic layer stabilizes said signal generating core; and
c) a water soluble outer shell, said water soluble outer shell comprising at least one of a polymer, a monomer, and a ligand, wherein said water soluble outer shell solubilizes each of said plurality of nanoparticles and provides biocompatibility for each of said plurality of nanoparticles.

2. The contrast agent of claim 1, wherein said signal generating core is superparamagnetic.

3. The contrast agent of claim 2, wherein said signal generating core comprises an iron oxide.

4. The contrast agent of claim 1, wherein said signal generating core has a diameter of up to about 30 nm.

5. The contrast agent of claim 4, wherein said signal generating core has a diameter in a range from about 4 nm to about 10 nm.

6. The contrast agent of claim 1, wherein said at least one organic layer comprises a water soluble surface binding polymer and a hydrophobic polymer.

7. The contrast agent of claim 1, wherein said at least one organic layer comprises a copolymer of a monomer having a pendant group and a hydrocarbon group, wherein said hydrocarbon group comprises a carbon chain of at least three carbon atoms.

8. The contrast agent of claim 7, wherein said at least one organic layer comprises at least one copolymer of acrylic acid, undecenoic acid, lauric acid, and combinations thereof.

9. The contrast agent of claim 8, wherein said at least one organic layer comprises at least one of polyacrylic acid, poly(undecenoic acid), poly(lauryl acrylate), and combinations thereof.

10. The contrast agent of claim 1, wherein said at least one organic layer comprises a monomer, said monomer having a surface binding head group and at least one polymerizable functionality.

11. The contrast agent of claim 10, wherein said monomer is undecenoic acid.

12. The contrast agent of claim 10, wherein said monomer is undecene trialkoxysilane.

13. The contrast agent of claim 1, wherein said at least one organic layer comprises a surfactant, said surfactant having a surface binding head group and at least one hydrocarbon tail.

14. The contrast agent of claim 13, wherein said surfactant is one of lauric acid and sodium dodecyl sulfate.

15. The contrast agent of claim 1, wherein said at least one organic layer has a thickness in a range from about 0.1 nm to about 100 nm.

16. The contrast agent of claim 1, wherein said water soluble outer shell further includes at least one targeting moiety.

17. The contrast agent of claim 16, wherein said at least one targeting moiety comprises at least one of a peptide, an antibody, a sugar, and combinations thereof.

18. The contrast agent of claim 17, wherein said at least one peptide comprises LSIPPKA.

19. The contrast agent of claim 17, wherein said at least one targeting moiety comprises one of folic acid and estradiol.

20. The contrast agent of claim 1, wherein said water soluble outer shell has a thickness in a range from about 0.1 nm to about 100 nm.

21. The contrast agent of claim 1, wherein said water soluble outer shell comprises a copolymer of a carboxylic acid and a hydrocarbon, said hydrocarbon having a carbon chain of at least three carbon atoms.

22. The contrast agent of claim 21, wherein said water soluble outer shell comprises at least one copolymer of acrylic acid, undecenoic acid, lauryl acrylate, and combinations thereof.

23. The contrast agent of claim 1, wherein said water soluble outer shell comprises a monomer, said monomer having a surface binding head group and at least one polymerizable functionality.

24. The contrast agent of claim 23, wherein said monomer is undecenoic acid.

25. The contrast agent of claim 1, wherein said water soluble outer shell comprises at least one ligand.

26. The contrast agent of claim 25, wherein said at least one ligand comprises a water soluble polymer attached to a hydrocarbon moiety, wherein said hydrocarbon moiety comprises a chain of at least tree carbon atoms.

27. The contrast agent of claim 26, wherein said hydrocarbon moiety further comprises at least one polymerizable functionality.

28. The contrast agent of claim 26, wherein said water soluble polymer is polyethylene glycol and said hydrocarbon moiety is undeceneoic acid.

29. The contrast agent of claim 1, wherein each of said plurality of nanoparticles has a diameter of up to about 100 nm.

30. The contrast agent of claim 29, wherein each of said plurality of nanoparticles has a diameter of up to about 50 nm.

31. The contrast agent of claim 30, wherein each of said plurality of nanoparticles has a diameter in a range from about 10 nm to about 30 nm.

32. A nanoparticle, said nanoparticle comprising:
a) a signal generating core, said signal generating core having a diameter of up to 10 nm;
b) a stabilizing coating disposed on and substantially covering said signal generating core, said stabilizing coating comprising:
i) an inner shell, said inner shell comprising at least one of a polymer, a monomer, and a surfactant, wherein said inner shell is adsorbed upon and substantially surrounds said signal generating core, and wherein said inner shell stabilizes said signal generating core; and
ii) a water soluble outer shell, said water soluble outer shell being disposed on an outer surface of said inner shell and substantially surrounding said inner shell, said water soluble outer shell comprising at least one of a second polymer, a second monomer, and a ligand, wherein said water soluble outer shell solubilizes said nanoparticle.

33. The nanoparticle of claim 32, wherein said signal generating core is superparamagnetic.

34. The nanoparticle of claim 33, wherein said signal generating core comprises an iron oxide.

35. The nanoparticle of claim 32, wherein said signal generating core further comprises at least one of gadolinium, manganese, copper, nickel, cobalt, zinc, germanium, gold, silver, II-VI compounds, IV-VI compounds, and combinations thereof.

36. The nanoparticle of claim 32, wherein said signal generating core is radio-opaque.

37. The nanoparticle of claim 36, wherein said signal generating core comprises at least one of gadolinium, and barium.

38. The nanoparticle of claim 32, wherein said signal generating core is responsive to laser radiation.

39. The nanoparticle of claim 38, wherein said signal generating core comprises at least one of gold, silver, and combinations thereof.

40. The nanoparticle of claim 32, wherein said signal generating core has a diameter of up to about 10 nm.

41. The nanoparticle of claim 40, wherein said signal generating core has a diameter in a range from about 4 nm to about 10 nm.

42. The nanoparticle of claim 32, wherein said inner shell comprises a water soluble surface binding polymer and a hydrophobic polymer.

43. The nanoparticle of claim 32, wherein said water soluble outer shell provides biocompatibility for said nanoparticle

44. The nanoparticle of claim 32, wherein said inner shell comprises a copolymer of a monomer having a pendant group and a hydrocarbon group, wherein said hydrocarbon group comprises a carbon chain of at least three carbon atoms.

45. The nanoparticle of claim 44, wherein said inner shell comprises at least one copolymer of acrylic acid, undecenoic acid, lauric acid, and combinations thereof.

46. The nanoparticle of claim 45, wherein said inner shell comprises polyacrylic acid, poly(undecenoic acid), poly(lauryl acrylate), and combinations thereof.

47. The nanoparticle of claim 32, wherein said inner shell comprises a monomer, said monomer having a surface binding head group and at least one polymerizable functionality.

48. The nanoparticle of claim 47, wherein said monomer is undecenoic acid.

49. The nanoparticle of claim 47, wherein said monomer is undecene trialkoxysilane.

50. The nanoparticle of claim 32, wherein said inner shell comprises a surfactant, said surfactant having a surface binding head group and at least one hydrocarbon tail.

51. The nanoparticle of claim 50, wherein said surfactant is one of lauric acid and sodium dodecyl sulfate.

52. The nanoparticle of claim 32, wherein said inner shell has a thickness in a range from about 0.1 nm to about 100 nm.

53. The nanoparticle of claim 32, wherein said water soluble outer shell further includes at least one targeting moiety.

54. The nanoparticle of claim 53, wherein said at least one targeting moiety comprises at least one of a peptide, an antibody, a nucleic acid, a sugar, and combinations thereof.

55. The nanoparticle of claim 54, wherein said at least one peptide comprises LSIPPKA.

56. The nanoparticle of claim 54, wherein said at least one targeting moiety comprises one of folic acid and estradiol.

57. The nanoparticle of claim 32, wherein said water soluble outer shell has a thickness in a range from about 0.1 nm to about 100 nm.

58. The nanoparticle of claim 32, wherein said water soluble outer shell comprises a copolymer of a carboxylic acid and a hydrocarbon, said hydrocarbon having a carbon chain of at least three carbon atoms.

59. The nanoparticle of claim 58, wherein said water soluble outer shell comprises at least one copolymer of acrylic acid, undecenoic acid, lauryl acrylate, and combinations thereof.

60. The nanoparticle of claim 32, wherein said water soluble outer shell comprises a monomer, said monomer having a surface binding head group and at least one polymerizable functionality.

61. The nanoparticle of claim 60, wherein said monomer is undecenoic acid.

62. The nanoparticle of claim 32, wherein said water soluble outer shell comprises at least one ligand.

63. The nanoparticle of claim 62, wherein said at least one ligand comprises a water soluble polymer attached to a hydrocarbon moiety, and wherein said hydrocarbon moiety comprises a chain of at least three carbon atoms.

64. The nanoparticle of claim 63, wherein said hydrocarbon moiety further comprises at least one polymerizable functionality.

65. The nanoparticle of claim 63, wherein said water soluble polymer is polyethylene glycol and said hydrocarbon moiety is undecenoic acid.

66. The nanoparticle of claim 32, wherein said nanoparticle has a diameter of up to about 100 nm.

67. The nanoparticle of claim 66, wherein said nanoparticle has a diameter of up to about 50 nm.

68. The nanoparticle of claim 67, wherein said nanoparticle has a diameter in a range from about 10 nm to about 30 nm.

69. A contrast agent, said contrast agent comprising a plurality of nanoparticles, wherein each of said plurality of nanoparticles comprises:
a) a signal generating core, said signal generating core having a diameter of up to 10 nm, wherein said signal generating core is superparamagnetic; and
b) a stabilizing coating disposed on and substantially covering said signal generating core, said stabilizing coating comprising:
i) an inner shell, said inner shell comprising at least one of a polymer, a monomer, and a surfactant, wherein said inner shell is adsorbed upon and substantially surrounds said signal generating core, and wherein said at least one organic layer stabilizes said signal generating core; and
ii) a water soluble outer shell, said water soluble outer shell disposed on an outer surface of said inner shell and substantially surrounding said inner shell, said water soluble outer shell comprising at least one of a second polymer, a second monomer, and a ligand wherein said water soluble outer shell solubilizes said nanoparticle and provides biocompatibility for said nanoparticle.

70. The contrast agent of claim 69, wherein said signal generating core comprises an iron oxide.

71. The contrast agent of claim 69, wherein said signal generating core has a diameter of up to about 30 nm.

72. The contrast agent of claim 69, wherein said signal generating core has a diameter in a range from about 4 nm to about 10 nm.

73. The contrast agent of claim 69, wherein said inner shell comprises a water soluble surface binding polymer and a hydrophobic polymer.

74. The contrast agent of claim 69, wherein said inner shell comprises a copolymer of a monomer having a pendant group and a hydrocarbon group, wherein said hydrocarbon group comprises a carbon chain of at least three carbon atoms.

75. The contrast agent of claim 74, wherein said inner shell comprises at least one copolymer of acrylic acid, undecenoic acid, lauric acid, and combinations thereof.

76. The contrast agent of claim 75, wherein said inner shell comprises at least one of polyacrylic acid, poly(undecenoic acid), lauryl acrylate, and combinations thereof.

77. The contrast agent of claim 69, wherein said inner shell comprises a monomer, said monomer having a surface binding head group and at least one polymerizable functionality.

78. The contrast agent of claim 77, wherein said monomer is undecenoic acid.

79. The contrast agent of claim 77, wherein said monomer is undecene trialkoxysilane.

80. The contrast agent of claim 69, wherein said inner shell comprises a surfactant, said surfactant having surface binding head group and at least one hydrocarbon tail.

81. The contrast agent of claim 80, wherein said surfactant is one of lauric acid and sodium dodecyl sulfate.

82. The contrast agent of claim 69, wherein said inner shell has a thickness in a range from about 0.1 nm to about 100 nm.

83. The contrast agent of claim 69, wherein said water soluble outer shell further includes at least one targeting moiety.

84. The contrast agent of claim 83, wherein said at least one targeting moiety comprises at least one of a peptide, an antibody, a nucleic acid, a sugar, and combinations thereof.

85. The contrast agent of claim 84, wherein said at least one peptide comprises LSIPPKA.

86. The contrast agent of claim 84, wherein said at least one targeting moiety comprises one of folic acid and estradiol.

87. The contrast agent of claim 69, wherein said water soluble outer shell has a thickness in a range from about 0.1 nm to about 100 nm.

88. The contrast agent of claim 69, wherein said water soluble outer shell comprises a copolymer of a carboxylic acid and a hydrocarbon, said hydrocarbon having a carbon chain of at least three carbon atoms.

89. The contrast agent of claim 88, wherein said water soluble outer shell comprises at least one copolymer of acrylic acid, undecenoic acid, lauryl acrylate, and combinations thereof.

90. The contrast agent of claim 89, wherein said at least one copolymer is one of polyacrylic acid, lauryl acrylate, and combinations thereof.

91. The contrast agent of claim 69, wherein said water soluble outer shell comprises a monomer, said monomer having an ionic head group and at least one polymerizable functionality.

92. The contrast agent of claim 91, wherein said monomer is undecenoic acid.

93. The contrast agent of claim 69, wherein said water soluble outer shell comprises at least one ligand.

94. The contrast agent of claim 93, wherein said at least one ligand comprises a water soluble polymer attached to a hydrocarbon moiety, wherein said hydrocarbon moiety comprises a chain of at least tree carbon atoms.

95. The contrast agent of claim 94, wherein said hydrocarbon moiety further comprises at least one polymerizable functionality.

96. The contrast agent of claim 94, wherein said water soluble polymer is polyethylene glycol and said hydrocarbon moiety is undecylene.

97. The contrast agent of claim 69, wherein each of said plurality of nanoparticles has a diameter of up to about 100 nm.

98. The contrast agent of claim 96, wherein each of said plurality of nanoparticles has a diameter of up to about 50 nm.

99. The contrast agent of claim 98, wherein each of said plurality of nanoparticles has a diameter in a range from about 10 nm to about 30 nm.

100. A method of making a plurality of monodisperse nanoparticles, wherein each of the plurality of nanoparticles comprises a plurality of a substantially crystalline signal generating core having a diameter of up to 10 nm, at least one polymerizable layer adsorbed upon and substantially surrounding the signal generating core, and a water soluble outer shell, the method comprising the steps of:
a) providing the signal generating core and the at least one polymerizable layer, wherein the at least one polymerizable layer is adsorbed upon and substantially surrounds the signal generating core, and wherein the at least one polymerizable layer stabilizes the signal generating core;
b) forming the water soluble shell on an outer surface of the at least one polymerizable layer, wherein the water soluble outer shell solubilizes and provides biocompatibility for each of the plurality of nanoparticles; and
c) covalently bonding the at least one polymerizable layer to the water soluble outer shell.

101. The method of claim 100, wherein the step of providing the signal generating core and the at least one polymerizable layer comprises:
a) combining a nonpolar aprotic organic solvent, an oxidant, and a first surfactant, wherein the first surfactant has a surface binding head group;
b) providing at least one organometallic compound to the combined nonpolar aprotic organic solvent, oxidant, and first surfactant, wherein the at least one organometallic compound comprises a metal and at least one ligand; and
c) heating the combined nonpolar aprotic organic solvent, oxidant, first surfactant, and the at least one organometallic compound under an inert gas atmosphere to a first temperature in a range from about 30° C. to about 400° C. for a first time interval to form the signal generating core surrounded by the first surfactant; and
d) precipitating the signal generating core surrounded by the first surfactant.

102. The method of claim 100, wherein the step of providing the signal generating core and the at least one polymerizable layer comprises:
a) combining deoxygenated water, Fe²⁺, and Fe³⁺ salts and heating to a temperature in a range from about 80° C. to about 90° C.;
b) providing at least one surfactant, monomer, ligand or polymer with surface adsorbing head group and aqueous alkali such as ammonium hydroxide; and
c) heating the combined aqueous solution of iron salts, base, and surface adsorbing monomer, surfactant, ligand or polymer, under an inert gas atmosphere at a temperature in a range from about 80° C. to about 100° C. to form the signal generating core surrounded by the first surfactant; and
d) isolating the signal generating core surrounded by the first surfactant.

103. The method of claim 100, wherein the signal generating core surrounded by the first surfactant is isolated by precipitation

104. The method of claim 100, wherein the signal generating core surrounded by the first surfactant is isolated by extracting with an organic solvent such as chloroform or toluene.

105. The method of claim 100, wherein the signal generating core is paramagnetic.

106. The method of claim 100, wherein the signal generating core is superparamagnetic.

107. The method of claim 100, wherein the signal generating core is responsive to laser radiation.

108. The method of claim 100, wherein the signal generating core is radioopaque.

109. The method of claim 100, wherein the step of providing the signal generating core and the at least one polymerizable layer comprises:
a) providing the signal generating core; and
b) adsorbing the at least one polymerizable layer onto a surface of the signal generating core.

110. The method of claim 100, wherein the at least one organic layer comprises at least one of a polymer, a monomer, a ligand, and a surfactant.

111. The method of claim 100, wherein the step of forming the water soluble shell on an outer surface of the at least one polymerizable layer comprises:
a) providing the signal generating cores coated with the at least one polymerizable layer in an organic solvent;
b) transferring the signal generating cores coated with the at least one polymerizable layer into an aqueous solution containing a material that forms the water soluble outer shell; and
c) transferring the signal generating cores into the aqueous phase, wherein the material adsorbs onto the at least one polymerizable layer to form the water soluble outer shell.

112. The method of claim 100, wherein the step of forming the water soluble shell on an outer surface of the at least one polymerizable layer comprises:
a) providing the signal generating cores coated with the at least one polymerizable layer;
b) transferring the signal generating cores coated with the at least one polymerizable layer into an aqueous solution containing a material that forms the water soluble outer shell; and
c) absorbing the material onto the at least one polymerizable layer to form the water soluble outer shell.

113. The method of claim 100, wherein the water soluble outer shell comprises at least one of a polymer, a monomer, and a ligand.

114. The method of claim 100, wherein the step of covalently bonding the at least one organic layer and the water soluble outer shell comprises polymerizing the at least one organic layer and the water soluble outer shell by at least one of heating and irradiation.

115. The method of claim 100, wherein the water soluble outer shell is polymerizable.

116. A nanoparticle, said nanoparticle comprising: >
a) a signal generating core, said signal generating core having a diameter of up to 10 nm;
b) an integrated coating comprising at least one of a polymer, a monomer, a ligand, and a surfactant, wherein said integrated coating is adsorbed upon and substantially surrounds said signal generating core, and wherein said integrated coating stabilizes said signal generating core and provides biocompatibility for said nanoparticle.
